SEQUENCE LISTING (2 sequences)

SEQ ID NO: 1 - protein, 31 aa, Homo sapiens
  MISC_FEATURE (30)..(30): Xaa at position 30 is Arg-NH2 when Xaa at position 31 is absent or is Arg
HAEGTFTSDVSSYLEGQAAKEFIAWLVKGXX

SEQ ID NO: 2 - protein, 39 aa, Heloderma sp.
  MISC_FEATURE (2)..(2): Xaa at position 2 is Ser or Gly
HXXGTFTSDLSKQMEEEAVRLFIEWLKNGGPSSGAPPPX

(19) United States
(12) Patent Application Publication
(10) Pub. No.: US 2005/0164925 A1
(43) Pub. Date: Jul. 28, 2005

(54) Treatment of gastroparesis

(75) Inventors: Joseph Anthony Jakubowski, Indianapolis, IN (US); Thurman Dwight McKinney, Indianapolis, IN (US)

Correspondence Address: ELI LILLY AND COMPANY;PATENT DIVISION, P.O. BOX 6288, INDIANAPOLIS, IN, 46206-6288, US

(73) Assignee: JOSEPH ANTHONY JAKUBOWSKI AND THURMAN DWIGHT MC KINNEY

(21) Appl. No.: 10/508,762

(22) PCT Filed: Mar. 27, 2003
(86) PCT No.: PCT/US03/08457

(30) Foreign Application Priority Data
Apr. 10, 2002 (US) 60371650

(51) Int. Cl. A61K 38/26

(52) U.S. Cl. 514/12

(57) ABSTRACT

The invention relates to the use of GLP-1 compounds to treat gastroparesis. Patients with gastroparesis generally experience a delay in gastric emptying. GLP-1 compounds can be used to regulate gastric emptying in these patients.

This invention relates to the use of glucagon-like peptide (GLP-1) compounds to treat gastroparesis. Gastroparesis, which means weak stomach, is a paralysis of the gastrointestinal (GI) system. It is a type of neuropathy causing stoppage or incorrect functioning of the autonomic nervous system resulting in delayed gastric emptying following ingestion of a meal. The stomach has two parts. The upper portion called the proximal stomach or fundus is where swallowed food and liquid collect. The lower portion called the distal stomach or antrum is where food is churned back and forth until it is broken into small fragments and then expelled into the duodenum which is the first part of the small intestine. Solid phase emptying is determined by powerful circular contractions of the antrum. In normal individuals, a coordinated wave of activity sweeps across the antrum about three times a minute following ingestion of a solid meal causing the stomach to contract. In individuals with gastroparesis, the electrical wave slows and the stomach contracts less frequently and sometimes with less force causing food to sit in the stomach.

Gastroparesis can be quite debilitating. Symptoms include nausea, vomiting, early satiety, abdominal bloating, epigastric pain or burning, and anorexia. Although gastroparesis occurs in non-diabetic patients, it is fairly common in patients with type 1 and type 2 diabetes. Approximately 75% of diabetics experience some type of gastrointestinal dysfunction and about 25 to 35% of diabetics have gastroparesis. It is not clear why the prevalence of this disease is so high in the diabetic population; however, it appears that glucose control is important since hyperglycemia causes delay in gastric emptying and exacerbates the symptoms associated with the disease.

Treatments currently available are not fully efficacious and are often associated with undesirable side effects. For example, prokinetic and antiemetic agents may be administered to treat delayed gastric emptying. McCallum, R. et al. Diabetic and nondiabetic gastroparesis: current treatment options 1 Gastroenterology 1-7 (1998). Intravenous erythromycin is often the treatment of choice for patients who cannot take oral medications due to the severity of the disease or other problems. However, erythromycin can cause GI toxicity, ototoxicity, pseudomembranous colitis, and the induction of resistant bacterial strains. For patients that can take oral medications, Cisapride is probably the most efficacious. Side effects of Cisapride include abdominal discomfort and increased frequency of bowel movements. In addition, there are important drug interactions that may cause heart arrhythmias; therefore, the drug is severely restricted as to its availability in the United States. There is clearly an unmet medical need regarding the treatment of this disease.

In diabetics, the severity of gastroparesis and the accompanying symptoms can be ameliorated to some extent with a strict insulin treatment regimen resulting in improved blood glucose control. However, gastroparesis and the associated delay in gastric emptying increases the risk of both hypoglycemia and hyperglycemia in diabetic patients treated with insulin or oral medications. When food delivery to the small bowel is interrupted due to gastroparesis, there is an elevated risk of hypoglycemia. The normal postprandial rise in blood glucose is delayed such that insulin administered before the meal reaches peak concentrations when blood glucose levels are still low. Blood glucose levels eventually rise hours later; however, at this point insulin concentrations have started to decline such that there is no longer enough insulin to counteract this hyperglycemia. Thus, in diabetic patients with moderate to severe gastroparesis, effective blood glucose control becomes nearly impossible due to the inability to predict when blood glucose levels will rise following a meal.

Treatment of gastroparesis using GLP-1 compounds solves the problems associated with the administration of compounds that are inefficacious and have severe side effects. In addition, GLP-1 has insulinotropic activity but does not cause hypoglycemia: thus, GLP-1 not only treats gastroparesis in diabetic patients but overcomes the problems associated with ineffective glucose control after treatment with insulin or oral agents in these patients.

GLP-1 analogs and derivatives are being developed primarily to treat type 2 diabetes. Although the insulinotropic effect of GLP-1 is well documented, the peptide has additional interesting physiological effects including causing delays in gastric emptying and inhibiting small bowel motility in rats. [Imeryuz, N. et al. Glucagon-like peptide-1 inhibits gastric emptying via vagal afferent-mediated central mechanisms, 273 Am. J. Physiol., G920-G927 (1997); Willms, B. et al. Gastric emptying, glucose responses, and insulin secretion after a liquid test meal: Effects of exogenous glucagons-like peptide-1 (GLP-1)-(7-36) Amide in type 2 (noninsulin dependent) diabetic patients, 81 J. Clin. Endocrinology, 327-332 (1996); Wettergren, A. et al. The inhibitory effect of glucagons-like peptide-1 (GLP-1) 7-36 amide on gastric acid secretion in humans depends on an intact vagal innervation, 40 Gut 597-601 (1997); Tolessa, T. et al. Inhibitoly effect of glucagons-like peptide-1 on small bowel motility, 102 J. Clin. Invest., 764-774 (1998). Further, GLP-1 has been implicated as a treatment for irritable-bowel syndrome (IBS) and functional dyspepsia. See U.S. Pat. No. 6,348,447 B1.

IBS and some types of functional dyspepsia are significantly different from gastroparesis. Functional dyspepsia is generally associated with chronic or recurrent pain centered in the abdomen. IBS stems from abnormal contractions of the colon which can affect the propulsion of stool and promote mixing and absorption of water. Hypermotility of the small intestine is also found and spasmodic cramping is a major source of pain. Although gastrointestinal motor function involves a complex interrelationship not completely understood between several events, it appears that IBS and possibly some types of functional dyspepsia are associated with problems in the lower GI tract whereas gastroparesis is associated with upper GI problems due to delayed gastric emptying. Given that GLP-1 has been shown to actually cause a delay in gastric emptying and inhibit smooth muscle contraction, it is surprising that the peptide can be used to treat gastroparesis which is a disorder thought to be caused by decreased contractility and delays in gastric emptying. This is an especially exciting discovery given that the insulinotropic action of GLP-1 is glucose dependent. Unlike the administration of insulin, there is not a risk of hypoglycemia associated with the administration of GLP-1. Thus, GLP-1 compounds which include GLP-1 analogs, GLP-1 derivatives, and agonists of the GLP-1 receptor can be used in diabetics to normalize blood glucose and in diabetics as well as non-diabetics to treat gastroparesis.

The present invention encompasses a method of treating gastroparesis in a patient suffering therefrom which comprises administering to said patient an effective amount of a GLP-1 compound or the use of a GLP-1 compound for the manufacture of a medicament for the treatment of gastroparesis. The patients with gastroparesis may be non-diabetic or diabetic patients. The GLP-1 compound may be administered by any method known to a person of ordinary skill in the art. Preferably, the GLP-1 compound is administered by subcutaneous injection, orally, or is absorbed through the buccal membrane.

FIG. 1: Graphs representing the mean (±SEM) glucose concentrations following once-daily administration of placebo (baseline), 2.5 mg (Group 1), and 3.5 mg (Group 2) of Val⁸-GLP-1 (7-37)OH to patients with type 2 diabetes.

FIG. 2: Graphs representing the mean (±SEM) glucose concentrations following once-daily administration of placebo (baseline) and 4.5 mg (Groups 3 and 4) of Val⁸-GLP-1(7-37)OH to patients with type 2 diabetes.

Methods and compositions using GLP-1 compounds are effective in treating gastroparesis. Gastroparesis is associated with defects in gastric emptying related to a dysregulation of stomach and pylorus contractility. GLP-1 compounds act to regulate stomach and/or pylorus contractility to diminish or eliminate delay in gastric emptying.

In 40% of the cases, gastroparesis has no known cause. The disease, however, occurs in approximately 25% to 35% of diabetics with one study finding the prevalence of the disorder to be as high as 59%. [Soykan, I. et al., Demography, clinical characteristics, psychological and abuse profile, treatment and long term follow-up of patients with gastroparesis, 11 Dig. Dis. Sci. 2398-2404 (1998); Hiba, R., Is there a difference in the prevalence of gastrointestinal symptoms between type 1 and type 2 diabetics? 4 Gastroenterology A79 (1999)].

Symptoms of gastroparesis include nausea, vomiting, postprandial bloating, epigastric pain, anorexia, and early satiety. In more severe cases, patients may vomit undigested food eaten a few hours before and may have a positive percussion splash sign along with signs of weight loss, dehydration, and malnutrition. Systemic causes of gastroparesis are evaluated by testing the patient for diabetes mellitus, hypothyroidism, cortisol deficiency, hypercalcemia, and pregnancy. Barium swallow, endoscopy, and upper GI series can rule out peptic ulcer disease and gastric outlet obstruction. Poor emptying of barium from the stomach may indicate slow gastric emptying. However, gastric scintigraphy is the gold standard for the proper diagnosis of gastroparesis. In this test, the patient is asked to eat a meal labeled with 99-M Technetium (TC) sulfur colloid or other radioactive ligand. The radioactivity is then measured in the stomach region using a gamma camera. The meal should be solid because emptying a liquid meal does not represent the actual gastric emptying. The results of the test can be reported as the time of emptying 50% of the meal or the percentage of emptying at specific intervals. [Thomforde, G. M. et al., Evaluation of an inexpensive screening scintigraphic test of gastric emptying, 36 J. Nucl. Med. 93 (1995)]. A breath test using 13-Carbon labeled food can also be used to measure gastric emptying. C¹³ is absorbed when it reaches the small bowel, and its measurements in the breath can indicate the gastric emptying. [Ghoos, Y. S., et al., 104 Gastroenterology 1640-1647 (1993)]. Electrogastrography (EGG) which measures electrical activity with cutaneous electrodes similar to those used in electrocardiograms can also be used to diagnose gastroparesis. [Stern, R. N. et al. EGG: Common issues in validation and methodology, 24 Psychophysiology 55-64 (1987)].

In normal gastric function when solid food reaches the stomach, the fundus relaxes and accommodates the ingested food. Gastric contractions mix and triturate the food in the antrum. Antral contractions mix and churn the food particles until they are less than 3 mm in size, a process that takes 30 to 40 minutes (the lag phase). Pyloric contractions that are coordinated with antral contractions move the particles into the duodenum. This complex sequence of coordinated events is controlled by an extrinsic nerve supply from the brain and spinal cord, complex neuronal networks within the wall of the stomach and intestine, and effects of locally released transmitters such as amines and peptides that alter the excitability of smooth muscle cells. Abnormalities in any of these networks can lead to abnormal gastric emptying and gastroparesis.

The ability of GLP-1 compounds to treat gastroparesis is surprising given the voluminous literature documenting the delay in gastric emptying that occurs when native GLP-1 is administered to humans. [See e.g. Willms, B., et al., Gastric emptying, glucose responses, and insulin secretion after a liquid test meal: effects of exogenous glucagons-like peptide (GLP-])-(7-36) amide in type 2 (noninsulin dependent) diabetic patients, 81 J. Clin. Endocrinology Metabolism, 327-332 (1996)]. The studies depicted in FIGS. 1 and 2 suggest that administration of a GLP-1 compound to type 2 diabetic patients without symptomatic gastroparesis does not delay gastric emptying compared to placebo. The time to peak glucose concentration following ingestion of a solid meal was identical for each group including the control group. GLP-1 compounds may not delay gastric emptying and instead normalize gastric emptying such that patients no longer experience one or more of the symptoms associated with gastroparesis.

Nitric oxide (NO) has been implicated as a neurotransmitter that affects the functioning of both the stomach and pylorus. Further, animal studies suggest that dysregulation of neuronal NO synthase (nNOS), the enzyme responsible for the production of NO, occurs in diabetics with gastrointestinal problems. [Watkins et al., Insulin restores neuronal nitric oxide synthase expression and function that is lost in diabetic gastropathy, 106 J. Clin. Invest. 373-384 (2000)]. The molecular mechanisms involved in expression of nNOS have not been elucidated. However, GLP-1 compounds may work in treating gastroparesis by indirectly regulating Nitric Oxide (NO) expression. Further, because some GLP-1 effects on GI function are lost in vagotomized humans, GLP-1 acts through neural pathways. This could involve GLP-1 receptors associated with vagal afferents, central nervous sites, or transmission in vagal efferents. In addition, GLP-1 receptors have been identified in the GI tract, mainly in the stomach and small intestine.

Although GLP-1 compounds can be used to treat gastroparesis in non-diabetic patients, the compounds are uniquely suited to treat gastroparesis in diabetic patients. GLP-1 compounds can regulate blood glucose levels by increasing insulin secretion and enhancing insulin sensitivity without causing hypoglycemia. GLP-1 compounds are, therefore, more effective in normalizing blood glucose levels in diabetic patients with gastroparesis because the compounds not only regulate gastric emptying in these patients but any unpredictable delays in gastric emptying associated with gastroparesis do not expose patients to an increased risk of hypoglycemia because the compounds do not cause hypoglycemia.

GLP-1 compounds appropriate for use in the present invention include native GLP-1, GLP-1 analogs, GLP-1 derivatives, Exendin-4, Exendin-4 analogs, Exendin-4 derivatives, and other agonists of the GLP-1 receptor.

GLP-1 analogs encompassed by the present invention have sufficient homology to GLP-1 (7-37)OH or a fragment of GLP-1 (7-37)OH such that the compound has the ability to bind to the GLP-1 receptor and initiate a signal transduction pathway resulting in the amelioration of one or more symptoms associated with gastroparesis. To determine whether a GLP-1 compound is suitable for the methods encompassed by the present invention an in vitro signaling assay can be used. Example 3 provides a table listing a number of GLP-1 analogs that have in vitro activity as measured by an assay that detects GLP-1 receptor signaling. Specifically, if a GLP-1 compound productively binds a GLP-1 receptor, the second messenger cAMP is activated. The extent of the induction of cAMP levels can then be measured using a cAMP response element which drives the expression of a reporter gene such as luciferase or beta lactamase.

The assay can be used to measure EC50 potency which is the effective concentration of GLP-1 compound that results in 50% activity in a single dose-response experiment. The assay is conducted using HEK-293 Aurora CRE-BLAM cells that stably express the human GLP-1 receptor. These HEK-293 cells have stably integrated a DNA vector having a cAMP response element (CRE) driving expression of the β-lactamase (BLAM) gene. The interaction of a GLP-1 agonist with the receptor initiates a signal that results in activation of the cAMP response element and subsequent expression of β-lactamase. The β-lactamase CCF2/AM substrate that emits flourescence when it is cleaved by β-lactamase (Aurora Biosciences Corp.) can then be added to cells that have been exposed to a specific amount of GLP-1 agonist to provide a measure of GLP-1 agonist potency. The assay is further described in Zlokarnik, et al. (1998) Science 279:84-88 (See also Example 3).

It is preferred that the GLP-1 compounds of the present invention have an in vitro potency no more than 10-fold lower, preferably no more than 5-fold lower, and more preferably no more than 3-fold lower than the in vitro potency of Val⁸-GLP-1(7-37)OH. Most preferably, the GLP-1 compounds have an in vitro potency not lower than the in vitro potency of Val⁸-GLP-1 (7-37)OH.

The biologically active forms of native GLP-1 are two truncated peptides known as GLP-1(7-37)OH and GLP-1 (7-36)amide. These two naturally occurring truncated GLP-1 peptides are represented in formula I, SEQ ID NO: 1.

SEQ ID NO:1

7 8 9 10 11 12 13 14 15 16 17
His-GLy-Glu-Gly-Thr-Phe-Thr-Ser-Asp-Val-Ser-
18 19 20 21 22 23 24 25 26 27 28
Ser-Tyr-Leu-Glu-Gly-Gln-Ala-Ala-Lys-Glu-Phe-
29 30 31 32 33 34 35 36 37
Ile-Ala-Trp-Leu-Val-Lys-Gly-Arg-Xaa
Formula I,

wherein:
Xaa at position 37 is Gly, or —NH₂.

Preferably, a GLP-1 compound has the amino acid sequence of SEQ ID NO:1 or is modified so that one, two, three, four or five amino acids differ from SEQ ID NO:1.

Some GLP-1 compounds known in the art include, for example, GLP-1(7-34) and GLP-1(7-35), GLP-1(7-36), Gln⁹-GLP-1(7-37), D-Gln⁹-GLP-1(7-37), Thr¹⁶-Lys¹⁸-GLP-1(7-37), and Lys¹⁸-GLP-1(7-37). GLP-1 compounds such as GLP-1(7-34) and GLP-1(7-35) are disclosed in U.S. Pat. No. 5,118,666. Other known biologically active GLP-1 analogs are disclosed in U.S. Pat. No. 5,977,071; U.S. Pat. No. 5,545,618; U.S. Pat. No. 5,705,483; U.S. Pat. No. 5,977,071; U.S. Pat. No. 6,133,235; Adelhorst, et al., J. Biol. Chem. 269:6275 (1994); and Xiao, Q., et al. (2001), Biochemistry 40:2860-2869.

GLP-1 compounds also include polypeptides in which one or more amino acids have been added to the N-terminus and/or C-terminus of GLP-1(7-37)OH, or fragments or analogs thereof. Preferably from one to eight amino acids are added to the C-terminus of GLP-1(7-37)OH. It is preferred that GLP-1 compounds of this type have up to about thirty-nine amino acids. The amino acids in the “extended” GLP-1 compounds are denoted by the same number as the corresponding amino acid in GLP- I (7-37)OH. For example, the N-terminal amino acid of a GLP-1 compound obtained by adding two amino acids to the N-terminus of GLP-1(7-37)OH is at position 5; and the C-terminal amino acid of a GLP-1 compound obtained by adding one amino acid to the C-terminus of GLP-1(7-37)OH is at position 38. Amino acids 38-45 of an extended GLP-1 compound are preferably the same as or a conservative substitution of the amino acid at the corresponding position of Exendin-3 or Exendin-4. The amino acid sequence of Exendin-3 and Exendin-4 are represented in formula II, SEQ ID NO: 2.

SEQ ID NO:2

7 8 9 10 11 12 13 14 15 16 17
His-Xaa-Xaa-Gly-Thr-Phe-Thr-Ser-Asp-Leu-Ser-
18 19 20 21 22 23 24 25 26 27 28
Lys-Gln-Met-Glu-Glu-Glu-Ala-Val-Arg-Leu-Phe-
29 30 31 32 33 34 35 36 37 38 39
Ile-Glu-Trp-Leu-Lys-Asn-Gly-Gly-Pro-Ser-Ser-
40 41 42 43 44 45
Gly-Ala-Pro-Pro-Pro-Ser

wherein:
Xaa at position 8 is Ser or Gly; and
Xaa at position 9 is Asp or Glu;
Ser at position 45 is Ser or Ser-NH2.

Exendin-3 has Ser at position 8 and Asp at position 9. Exendin-4 has Gly at position 8 and Glu at position 9.

GLP-1 compounds that are most preferred comprise GLP-1 analogs wherein the backbone for such analogs or fragments contains an amino acid other than alanine at position 8 (position 8 analogs). Preferred amino acids at position 8 are glycine, valine, leucine, isoleucine, serine, threonine, or methionine and more preferably are valine or glycine.

Other preferred GLP-1 compounds are GLP-1 analogs that have the sequence of GLP-1 (7-37)OH except that the amino acid at position 8 is preferably glycine, valine, leucine, isoleucine, serine, threonine, or methionine and more preferably valine or glycine and position 22 is glutamic acid, lysine, aspartic acid, or arginine and more preferably glutamic acid or lysine.

Other preferred GLP-1 compounds are GLP-1 analogs that have the sequence of GLP-1(7-37)OH except that the amino acid at position 8 is preferably glycine, valine, leucine, isoleucine, serine, threonine, or methionine and more preferably valine or glycine and position 30 is glutamic acid, aspartic acid, serine, or histidine and more preferably glutamic acid.

Other preferred GLP-1 compounds are GLP-1 analogs that have the sequence of GLP-1(7-37)OH except that the amino acid at position 8 is preferably glycine, valine, leucine, isoleucine, serine, threonine, or methionine and more preferably valine or glycine and position 37 is histidine, lysine, arginine, threonine, serine, glutamic acid, aspartic acid, tryptophan, tyrosine, phenylalanine and more preferably histidine.

Other preferred GLP-1 compounds are GLP-1 analogs that have the sequence of GLP-1(7-37)OH except that the amino acid at position 8 is preferably glycine, valine, leucine, isoleucine, serine, threonine, or methionine and more preferably valine or glycine and position 22 is glutamic acid, lysine, aspartic acid, or arginine and more preferably glutamic acid or lysine and position 27 is alanine, lysine, arginine, tryptophan, tyrosine, phenylalanine, or histidine and more preferably alanine.

In the nomenclature used herein to describe GLP-1 compounds, the substituting amino acid and its position is indicated prior to the parent structure. For example Val⁸-GLP-1(7-37)OH designates a GLP-1 compound in which the alanine normally found at position 8 in GLP-1(7-37)OH (formula I, SEQ ID NO:1) is replaced with valine.

Other preferred GLP-1 compounds include: Val⁸-GLP-1(7-37)OH, Gly⁸-GLP-1(7-37)OH, Glu²²-GLP-1(7-37)OH, Asp²²-GLP-1(7-37)OH, Arg²²-GLP-1(7-37)OH, Lys²²-GLP-1(7-37)OH, Cys²²-GLP-1(7-37)OH, Val⁸-Glu²²-GLP-1(7-37)OH, Val⁸-Asp²²-GLP-1(7-37)OH, Val⁸-Arg²²-GLP-1(7-37)OH, Val⁸-Lys²²-GLP-1(7-37)OH, Val⁸-Cys²²-GLP-1(7-37)OH, Gly⁸-Glu²²-GLP-1(7-37)OH, Gly⁸-Asp²²-GLP-1(7-37)OH, Gly⁸-Arg²²-GLP-1(7-37)OH, Gly⁸-Lys²²-GLP-1(7-37)OH, Gly⁸-Cya²²-GLP-1(7-37)OH, Glu²²-GLP-1 (7-36)NH₂, Asp²²-GLP-1(7-36)NH₂, Arg²²-GLP-1(7-36)NB₂, Lys²²-GLP-1(7-36)NH₂, Cys²²-GLP-1(7-36)NH₂, Val⁸-Glu²²-GLP-1(7-36)NH₂, Val⁸ Asp²²-GLP-1(7-36)NH₂, Val⁸-Arg²²-GLP-1(7-36)NH₂, Val⁸-Lys²²-GLP-1(7-36)NH₂, Val⁸-Cys²²-GLP-1(7-36)NH₂, Gly⁸-Glu²²-GLP-1(7-36)NH₂, Gly⁸-Asp²²-GLP-1(7-36)NH₂, Gly⁸-Arg²²-GLP-1(7-36)NH₂, Gly⁸-Lys²²-GLP-1(7-36)NH₂, Gly⁸-Cys²²-GLP-1(7-36)NH₂, Lys²³-GLP-1(7-37)OH, Val⁸-Lys²³-GLP-1(7-37)OH, Gly⁸-Lys²³-GLP-1(7-37)OH, His²⁴-GLP-1(7-37)OH, Val⁸-His²⁴-GLP-1(7-37)OH, Gly⁸-His²⁴-GLP-1(7-37)OH, Lys²⁴-GLP-1(7-37)OH, Val⁸-Lys²⁴-GLP-1(7-37)OH, Gly⁸-Lys²⁴-GLP-1(7-37)OH, Glu³⁰-GLP-1(7-37)OH, Val⁸-Glu³⁰-GLP-1(7-37)OH, Gly⁸-Glu³⁰-GLP-1(7-37)OH, Asp³⁰-GLP-1(7-37)OH, Val⁸-Asp³⁰-GLP-1(7-37)OH, Gly⁸-Asp³⁰-GLP-1(7-37)OH, Gln³⁰-GLP-1(7-37)OH, Val⁸-Gln³⁰-GLP-1(7-37)OH, Gly⁸-Gln³⁰-GLP-1(7-37)OH, Tyr³⁰-GLP-1(7-37)OH, Val⁸-Tyr³⁰-GLP-1(7-37)OH, Gly⁸-Tyr³⁰-GLP-1(7-37)OH, Ser³⁰-GLP-1(7-37)OH, Val⁸-Ser³⁰-GLP-1(7-37)OH, Gly⁸-Ser³⁰-GLP-1(7-37)OH, His³⁰-GLP-1(7-37)OH, Val⁸-His³⁰-GLP-1(7-37)OH, Gly⁸-His³⁰-GLP-1(7-37)OH, Glu³⁴-GLP-1(7-37)OH, Val⁸-Glu³⁴-GLP-1(7-37)OH, Gly⁸-Glu³⁴-GLP-1(7-37)OH, Ala³⁴-GLP-1(7-37)OH, Val⁸-Ala³⁴-GLP-1(7-37)OH, Gly⁸-Ala³⁴-GLP-1(7-37)OH, Gly³⁴-GLP-1(7-37)OH, Val⁸-Gly³⁴-GLP-1(7-37)OH, Gly⁸-Gly³⁴-GLP-1(7-37)OH, Ala³⁵-GLP-1(7-37)OH, Val⁸-Ala³⁵-GLP-1(7-37)OH, Gly⁸-Ala³⁵-GLP-1(7-37)OH, Lys³⁵-GLP-1(7-37)OH, Val⁸-Lys³⁵-GLP-1(7-37)OH, Gly⁸-Lys³⁵-GLP-1(7-37)OH, His³⁵-GLP-1(7-37)OH, Val⁸-His³⁵-GLP-1(7-37)OH, Gly⁸-His³⁵-GLP-1(7-37)OH, Pro³⁵-GLP-1(7-37)OH, Val⁸-Pro³⁵-GLP-1(7-37)OH, Gly⁸-Pro³⁵-GLP-1(7-37)OH, Glu³⁵-GLP-1(7-37)OH, Val⁸-Glu³⁵-GLP-1(7-37)OH, Gly⁸-Glu³⁵-GLP-1(7-37)OH, Val⁸-Ala²⁷-GLP-1(7-37)OH, Val⁸-His³⁷-GLP-1(7-37)OH, Val⁸-Glu²²-Lys²³-GLP-1(7-37)OH, Val⁸-Glu²²-Glu²³-GLP-1(7-37)OH, Val⁸-Glu²²-Ala²⁷-GLP-1(7-37)OH, Val⁸-Gly³⁴-Lys³⁵-GLP-1(7-37)OH, Val⁸-His³⁷-GLP-1(7-37)OH, and Gly⁸-His³⁷-GLP-1(7-37)OH.

More preferred GLP-1compounds are Val⁸-GLP-1(7-37)OH, Gly⁸-GLP-1(7-37)OH, Glu²²-GLP-1(7-37)OH, Lys²²-GLP-1(7-37)OH, Val⁸-Glu²²-GLP 1(7-37)OH, Val⁸-Lys²²-GLP-1(7-37)OH, Gly⁸-Glu²²-GLP-1(7-37)OH, Gly⁸-Lys²²-GLP-1(7-37)OH, Glu²²-GLP-1(7-36)NH₂, Lys²²-GLP-1(7-36)NH₂, Val⁸-Glu²²-GLP-1(7-36)NH₂, Val⁸-Lys²²-GLP-1(7-36)NB₂, Gly⁸-Glu²²-GLP-1(7-36)NH₂, Gly⁸-Lys²²-GLP-1(7-36)NH₂, Val⁸-His³⁷-GLP-1(7-37)OH, Gly⁸-His³⁷-GLP-1(7-37)OH, Arg³⁴-GLP-1(7-36)NH₂, and Arg³⁴-GLP-1(7-37)OH.

Other preferred GLP-1 compounds include: Val⁸-Tyr¹²-GLP-1(7-37)OH, Val⁸-Tyr¹²-GLP-1(7-36)NH₂, Val⁸-Trp¹²-GLP-1(7-3 7)OH, Val⁸-Leu¹⁶-GLP-1(7-37)OH, Val⁸-Tyr -GLP-1(7-37)OH, Gly⁸-Glu²² -GLP-1(7-37)OH, Val⁸-Leu²⁵-GLP-1(7-37)OH, Val⁸-Glu³⁰-GLP-1(7-37)OH, Val⁸-His³⁷-GLP-1(7-37)OH, Val⁸-Tyr¹²-Tyr¹⁶-GLP-1(7-37)OH, Val⁸-Trp¹²-Glu²²-GLP-1(7-37)OH, Val⁸-Tyr 2-Glu²² -GLP-1(7-37)OH, Val⁸-Tyr¹⁶-Phe¹⁹-GLP-1(7-37)OH, Val⁸-Tyr¹⁶-Glu²²-GLP-1(7-37)OH, Val⁸-Trp¹⁶-Glu²²-GLP-1(7-3 7)OH, Val⁸-Leu¹⁶-Glu²²-GLP-1(7-3 7)OH, Val⁸-Ile¹⁶-Glu²²-GLP-1(7-37)OH, Val⁸-Phe¹⁶-Glu²²-GLP-1(7-37)OH, Val⁸-Trp¹⁸-Glu²²-GLP-1(7-37)OH, Val⁸-Tyr¹⁸-Glu²²-GLP-1(7-37)OH, Val⁸-Phe¹⁸-Glu²²-GLP-1(7-37)OH, Val⁸-Ile¹⁸-Glu²²-GLP-1(7-37)OH, Val⁸-Lys¹⁸-Glu²²-GLP-1(7-37)OH, Val⁸-Trp¹⁹-Glu²²-GLP-1(7-37)OH, Val⁸-Phe¹⁹-Glu²²-GLP-1(7-37)OH, Val⁸-Phe²⁰-Glu²²-GLP-1(7-37)OH, Val⁸-Glu²²-Leu²⁵-GLP-1(7-37)OH, Val⁸-Glu²²-Ile²⁵GLP-1(7-37)OH, Val⁸-Glu²²-Val²⁵-GLP-1(7-37)OH, Val⁸-Glu²²-Ile²⁷-GLP-1(7-3 7)OH, Val⁸-Glu²²-Ala²⁷-GLP-1(7-37)OH, Val⁸-Glu²²-Ile³³-GLP-1(7-37)OH, Val⁸-Glu²²-His³⁷-GLP-1(7-37)OH, Val⁸-Asp⁹-Ile¹¹-Tyr¹⁶-Glu²²-GLP-1(7-37)OH, Val⁸-Tyr¹⁶-Trp¹⁹-Glu²²-GLP-1(7-37)OH, Val⁸-Trp¹⁶-Glu²²-Val²⁵-Ile³³-GLP-1(7-37)OH, Val⁸-Trp¹⁶-Glu²²-Ile³³ -GLP-1(7-37)OH, Val⁸-Glu²²-Val²⁵-Ile³³-GLP-1(7-37)OH, and Val⁸-Trp¹⁶-Glu²²-Val²⁵-GLP-1(7-37)OH.

A GLP-1 compound also includes a “GLP-1 derivative” which is defined as a molecule having the amino acid sequence of native GLP-1, Exendin-4, or of a GLP-1 or Exendin-4 analog, but additionally having chemical modification of one or more of its amino acid side groups, α-carbon atoms, terminal amino group, or terminal carboxylic acid group. A chemical modification includes, but is not limited to, adding chemical moieties, creating new bonds, and removing chemical moieties.

Modifications at amino acid side groups include, without limitation, acylation of lysine ε-amino groups, N-alkylation of arginine, bistidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist. The ax-carbon of an amino acid may be mono- or dimethylated.

Preferred GLP-1 derivatives are achieved through acylation. Using the principle of fatty acid derivitization, GLP-1 action is protracted by facilitating binding to plasma albumin via association of the fatty acid residue to fatty acid binding sites on albumin in the blood and peripheral tissues. A preferred GLP-1 derivative is Arg³⁴Lys²⁶-(N-ε(γ-Glu(N-α-hexadecanoyl)))-GLP-1(7-37). GLP-1 derivatives and methods of making such derivatives are disclosed in Knudsen et al. (2000) J. Med. Chem. 43:1664-1669. In addition, numerous published applications describe derivatives of GLP-1, GLP-1 analogs, Exendin-4, and Exendin-4 analogs. See U.S. Pat. No. 5,512,540, U.S. Pat. No. 6,268,343, WO96/29342, WO98/08871, WO99/43341, WO99/43708, WO99/43707, WO99/43706, and WO99/43705.

GLP-1 compounds that are protected from degradation by the endogenous protease dipeptidyl-peptidase IV (DPP-IV) are most preferred. DPP-IV cleaves after the N-terminal histidine residue of native truncated GLP-1 and inactivates the molecule. Native GLP-1 has a half-life of approximately 5 to 10 minutes due to rapid inactivation by DPP-IV. Resistance of a particular GLP-1 compound to DPP-IV is determined by incubating the GLP-1 compound in human plasma. For example, human plasma is incubated at 37° C. with a 300 pmolar solution of a GLP-1 compound for up to six hours. The solution is then subjected to reversed phase HPLC and RIA according to Deacon et al., 80 J. Clin. Endocrinol. Metab. 952-957 (1995). Position 8 analogs are protected from DPP-IV activity as are some GLP-1 derivatives such as acylated GLP-1 analogs wherein the bulky acyl groups prevents DPP-IV from binding to the N-terminus of the analog.

GLP-1 compounds can be made by a variety of methods known in the art such as solid-phase synthetic chemistry, purification of GLP-1 molecules from natural sources, recombinant DNA technology, or a combination of these methods. For example, methods for preparing GLP-1 compounds are described in the following U.S. Pat. Nos.: 5,118,666; 5,120,712; 5,512,549; 5,977,071; and 6,191,102.

The GLP-1 compounds of the present invention maybe formulated as pharmaceutically acceptable compositions. “Pharmaceutically acceptable” means suitable for administration to a human. A pharmaceutically acceptable formulation does not contain toxic elements, undesirable contaminants or the like, and does not interfere with the activity of the active compounds therein. Pharmaceutically acceptable compositions of the present invention may take various forms, such as, for example, powders, granules, tablets, sugar-coated tablets, capsules, syrups, suppositories, injectable solutions, preparations for inhalation, preparations for nasal administration, or preparations for buccal administration. Depending on the form of pharmaceutically acceptable compositions comprised of a GLP-1 compound, such compositions may be administered by a variety of routes such as orally, by nasal administration, by buccal administration, by inhalation, or parenterally. Parenteral administration can include, for example, systemic administration, such as by intramuscular, intravenous, subcutaneous, or intraperitoneal injection. [See Gutniak et al., GLP-1 tablet in type 2 diabetes in fasting and postprandial conditions, 20 Diabetes Care 1874-1879 (1997); Gutniak, et al., Potential therapeutic levels of glucagon-like peptide I achieved in humans by a buccal tablet, 19 Diabetes Care 843-848 (1996)].

A pharmaceutically acceptable drug product may have the GLP-1 compound combined with a pharmaceutically-acceptable buffer, wherein the pH is suitable for parenteral administration and adjusted to provide acceptable stability and solubility properties. Pharmaceutically-acceptable anti-microbial agents may also be added. Meta-cresol and phenol are preferred pharmaceutically-acceptable anti-microbial agents. One or more pharmaceutically-acceptable salts may also be added to adjust the ionic strength or tonicity. One or more excipients may be added to further adjust the isotonicity of the formulation. Glycerin is an example of an isotonicity-adjusting excipient.

GLP-1 compounds may be administered on an as needed basis or may be administered chronically. On an as needed basis refers to acute or on-demand administration. For example, especially in non-diabetic patients with gastroparesis, it may be preferable to administer a GLP-1 compound that is relatively short acting immediately before or after a meal to reduce or eliminate symptoms associated with gastroparesis. Further, gastroparesis and associated symptoms may be worse at a particular time of day or the severity of the disease and associated symptoms may vary on a day to day, week to week, or even a month to month basis. Thus, a patient may receive a GLP-1 compound as needed, for example, only when symptoms are uncomfortable.

For diabetic patients with gastroparesis, it may be preferable to administer GLP-1 compounds chronically. “Chronic” generally refers to regular administration for an extended period preferably not more frequently than four times daily, most preferably not more than two or three times daily, even more preferably not more than once daily. However, chronic administration as used herein may encompass other regimens in addition to daily dosing. For example, chronic administration encompasses administration of a sustained release formulation that provides sufficient therapeutic blood plasma levels on a regular basis. Such administration may include administration once a week, once a month, or even less frequently.

It is preferable, especially for chronic therapy, that GLP-1 compounds are derivatized or formulated such that they have a protracted profile of action. For example, GLP-1 analogs such as position 8 analogs are resistant to DPP-IV cleavage and have a protracted profile of action. In addition, acylated GLP-1 derivatives have a protracted profile of action due to their albumin binding properties. GLP-1 analogs can be complexed with zinc and/or protamine and formulated as a suspension to provide a protracted profile of action. For example, see WO99/30731 wherein GLP-1 compound crystallization conditions are described.

An “effective amount” of a GLP-1 compound is the quantity that results in a desired effect without causing unacceptable side-effects when administered to a subject. A desired effect can include an amelioration of symptoms associated with gastroparesis. In particular, the desired effect is the regulation of gastric emptying such that gastroparesis is eliminated. For chronic administration, to achieve efficacy while minimizing side effects, the plasma levels of a GLP-1 compound should not fluctuate significantly once steady state levels are obtained during the course of treatment. Levels do not fluctuate significantly if they are maintained within the ranges described herein once steady state levels are achieved throughout a course of treatment. Most preferably, plasma levels of a GLP-1 compound with a potency similar to or within two-fold that of Val⁸-GLP-1(7-37)OH are maintained between about 30 picomolar and about 200 picomolar, preferably between about 60 picomolar and about 150 picomolar throughout a course of treatment once steady state levels are obtained.

The optimal range of plasma levels appropriate for Val⁸-GLP-1(7-37)OH and GLP-1 compounds of similar potency can also be applied to other GLP-1 compounds including Exendin-3 and Exendin-4 which have different potencies. GLP-1 compounds of similar potency include compounds that have within two-fold the activity of Val⁸-GLP-1(7-37)OH as measured by the in vitro potency assay described in Example 3.

Exendin-4 has a potency that is approximately 5-fold higher than Val⁸-GLP-1(7-37)OH; thus, optimum plasma levels of Exendin-4 will be approximately 5-fold lower than the levels appropriate for Val⁸-GLP-1(7-37)OH and compounds of similar potency. This would correspond to plasma levels in the range between about 6 picomolar and about 40 picomolar, preferably between about 12 picomolar and about 30 picomolar. Another example of a GLP-1 compound with increased potency is Val⁸-Glu²²-GLP-1(7-37)OH which has a potency approximately 3-fold higher than Val⁸-GLP-1(7-37)OH. Thus, optimum plasma levels of this compound will be approximately 3-fold lower than the levels determined for Val⁸-GLP-1(7-37)OH.

The invention also encompasses an article of manufacture for human pharmaceutical use comprising a container; a dosage form comprising an amount of a GLP-1 compound such as a GLP-1 analog or derivative and a package insert providing that administration of the dosage form results in the alleviation of symptoms associated with gastroparesis or the effective treatment of gastroparesis.

“Container” means any receptacle and closure suitable for storing, shipping, dispensing, and/or handling a pharmaceutical product.

“Packaging” means a customer-friendly device allowing convenient administration and/or ancillary devices that aid in delivery, education, and/or administration. The packaging may improve GLP-1 compound administration to the patient, reduce or improve educational instruction time for the patient, provide a platform for improved health economic studies, and/or limit distribution channel workload. Also, the packaging may include but not be limited to a paper-based package, shrink wrapped package, see-through top packaging, trial-use coupons, educational materials, ancillary supplies, and/or delivery device.

“Package insert” means information accompanying the product that provides a description of how to administer the product, along with the safety and efficacy data required to allow the physician, pharmacist, and patient to make an informed decision regarding use of the product, and/or patient education information. The package insert generally is regarded as the “label” for a pharmaceutical product.

Thus, the present invention encompasses articles of manufacture for human pharmaceutical use comprising a package insert and a container wherein said insert describes a treatment regimen which involves administering a dosage form to treat gastroparesis. The effective treatment of gastroparesis encompasses the alleviation or elimination of one or more symptoms associated with gastroparesis or the elimination of all symptoms associated with gastroparesis. The effective treatment of gastroparesis also encompasses a normalization in gastric function which may include gastric emptying following a meal.

The container used in the present article of manufacture is conventional in the pharmaceutical arts. Generally, the container is a vial or cartridge, usually made of glass, and accompanying cap, closure, bead, plunger, septum, and/or seal or other such article suitable for use by the patient or pharmacist. Alternatively, the container is part of a kit consisting of a cartridge containing dried powder and a syringe pre-filled with an appropriate diluent. Other options include the container consisting of a dual chamber cartridge with a bypass that keeps the diluent liquid and the dried powder separate from each other until reconstitution is desired. At the time of reconstitution, the dual chamber cartridge permits the diluent liquid to flow into the dried powder. Preferably, the container is sized to accommodate 0.1 to 100 mL, preferably 0.5 to 25 mL, and more preferably, 5 to 10 mL, even more preferably 1.5 to 3 mL volumes. Alternatively, the container is a blister, capsule, or blister disc. Other options for the container include a transdermal patch, implantable device, microsphere carriers and other depot delivery systems.

The insert may provide the physician with a choice of several doses which result in specific plasma levels of the GLP-1 compound. Preferable ranges are described herein. Preferably the insert will provide the physician with a single dose which results in plasma levels of the GLP-1 compound within the ranges described herein.

The package insert provides a description of how to administer a pharmaceutical product, along with the safety and efficacy data required to allow the physician, pharmacist, and patient to make an informed decision regarding the use of the product. The package insert generally is regarded as the label of the pharmaceutical product.

The package insert may provide some or all of the following indications or label descriptions: a) use in patients with gastroparesis; b) use in diabetic patients with gastroparesis; c) improved glycemic control in diabetic patients with gastroparesis; and d) no symptomatic hypoglycemia at dose effective to eliminate or reduce the severity of one or more symptoms associated with gastroparesis.

### EXAMPLE 1

### Clinical Study in Type 2 Diabetics

Four groups with eight type 2 diabetic patients in each group were treated with a long-acting formulation of Val⁸-GLP-1(7-37)OH. The first three groups received either 2.5 or 3.5 or 4.5 mg by subcutaneous injection once a day for 6 days. The fourth group received 4.5 mg by subcutaneous injection once per day for 21 days. On the day before the study, each patient received a saline injection as placebo. Blood glucose was followed for 13 hours. All meals during the evaluation days were strictly standardized. Patients were outpatients except for the Day 6 and Day 21 evaluations over 24 hours. Following the injection on Day 1, blood samples were taken for glucose and Val⁸-GLP-1(7-37)OH plasma levels during 4 hours. Patients were dosed each morning. On the sixth day of dosing (and also Day 21 for Group 4), samples were collected up to 26 hours post dose for the blood glucose and Val⁸-GLP-1(7-37)OH plasma level determinations. Glucose levels are represented in FIGS. 1 and 2.

### EXAMPLE 2

### Determination of Val8-GLP-1(7-37)OH Plasma Levels

Due to the presence of endogenous concentrations of native GLP-1 peptides and degradation products such as GLP-1(9-37)OH by DPP-IV, concentrations of intact Val⁸-GLP-1(7-37)OH were measured using an ELISA assay in which full-length non-degraded Val⁸-GLP-1(7-37)OH is specifically recognized. Immunoreactive Val⁸-GLP-1(7-37)OH is captured from the plasma by an N-terminal anti-Val⁸-GLP-1(7-37)OH specific antisera immobilized onto a microtiter plate. This antisera is highly specific to the N-terminus of Val⁸-GLP-1(7-37)OH. An alkaline-phosphatase conjugated antibody, specific for the C-terminus of GLP-1, is added to complete the “sandwich.” Detection is completed using pNPP, a colormetric substrate for alkaline phosphatase. The amount of color generated is directly proportional to the concentration of immunoreactive Val⁸-GLP-1(7-37)OH present in the sample. Quantitation of Val⁸-GLP-1(7-37)OH in human plasma can be interpolated from a standard curve using Val⁸-GLP-1(7-37)OH as the reference standard. Data was analyzed by a computer program using a weighted 4-parameter logistic algorithm. The concentration of immunoreactive Val⁸-GLP-1(7-37)OH in test samples was determined using a standard curve.

### EXAMPLE 3

### In Vitro Potency Assay

HEK-293 Aurora CRE-BLAM cells expressing the human GLP-1 receptor are seeded at 20,000 to 40,000 cells/well/100 μl into a 96 well black clear bottom plate. The day after seeding, the medium is replaced with plasma free medium. On the third day after seeding, 20 μl of plasma free medium containing different concentrations of GLP-1 agonist is added to each well to generate a dose response curve. Generally, fourteen dilutions containing from 3 nanomolar to 30 nanomolar GLP-1 compound were used to generate a dose response curve from which EC₅₀ values could be determined. After 5 hours of incubation with GLP-1 compound, 20 μl of β-lactamase substrate (CCF2-AM —Aurora Biosciences—product code 100012) was added and incubation continued for 1 hour at which point the flourescence was determined on a cytoflour.

TABLE 1

| | EC₅₀ Relative to |
|---|---|
| Compound | Val⁸-GLP-1(7-37)OH |
| Val⁸-GLP-1(7-37)OH | 1.0 |
| Gly⁸-GLP-1(7-37)OH | 1.7 |
| Val⁸-Tyr¹²-GLP-1(7-37)OH | 1.7 |
| Val⁸-Tyr¹²-GLP-1(7-36)NH₂ | 1.1 |
| Val⁸-Trp¹²-GLP-1(7-37)OH | 1.1 |
| Val⁸-Leu¹⁶-GLP-1(7-37)OH | 1.1 |
| Val⁸-Tyr¹⁶-GLP-1(7-37)OH | 2.5 |
| Gly⁸-Glu²²-GLP-1(7-37)OH | 2.2 |
| Val⁸-Leu²⁵-GLP-1(7-37)OH | 0.5 |
| Val⁸-Glu³⁰-GLP-1(7-37)OH | 0.7 |
| Val⁸-His³⁷-GLP-1(7-37)OH | 1.2 |
| Val⁸-Tyr¹²-Tyr¹⁶-GLP-1(7-37)OH | 1.5 |
| Val⁸-Trp¹²-Glu²²-GLP-1(7-37)OH | 1.7 |
| Val⁸-Tyr¹²-Glu²²-GLP-1(7-37)OH | 2.7 |
| Val⁸-Tyr¹⁶-Phe¹⁹-GLP-1(7-37)OH | 2.8 |
| Val⁸-Tyr¹⁶-Glu²²-GLP-1(7-37)OH | 3.6, 3.8 |
| Val⁸-Trp¹⁶-Glu²²-GLP-1(7-37)OH | 4.9, 4.6 |
| Val⁸-Leu¹⁶-Glu²²-GLP-1(7-37)OH | 4.3 |
| Val⁸-Ile¹⁶-Glu²²-GLP-1(7-37)OH | 3.3 |
| Val⁸-Phe¹⁶-Glu²²-GLP-1(7-37)OH | 2.3 |
| Val⁸-Trp¹⁸-Glu²²-GLP-1(7-37)OH | 3.2, 6.6 |
| Val⁸-Tyr¹⁸-Glu²²-GLP-1(7-37)OH | 5.1, 5.9 |
| Val⁸-Phe¹⁸-Glu²²-GLP-1(7-37)OH | 2.0 |
| Val⁸-Ile¹⁸-Glu²²-GLP-1(7-37)OH | 4.0 |
| Val⁸-Lys¹⁸-Glu²²-GLP-1(7-37)OH | 2.5 |
| Val⁸-Trp¹⁹-Glu²²-GLP-1(7-37)OH | 3.2 |
| Val⁸-Phe¹⁹-Glu²²-GLP-1(7-37)OH | 1.5 |
| Val⁸-Phe²⁰-Glu²²-GLP-1(7-37)OH | 2.7 |
| Val⁸-Glu²²-Leu²⁵-GLP-1(7-37)OH | 2.8 |
| Val⁸-Glu²²-Ile²⁵-GLP-1(7-37)OH | 3.1 |
| Val⁸-Glu²²-Val²⁵-GLP-1(7-37)OH | 4.7, 2.9 |
| Val⁸-Glu²²-Ile²⁷-GLP-1(7-37)OH | 2.0 |
| Val⁸-Glu²²-Ala²⁷-GLP-1(7-37)OH | 2.2 |
| Val⁸-Glu²²-Ile³³-GLP-1(7-37)OH | 4.7, 3.8, 3.4 |
| Val⁸-Glu²²-His³⁷-GLP-1(7-37)OH | 4.7 |
| Val⁸-Asp⁹-Ile¹¹-Tyr¹⁶-Glu²²-GLP-1(7-37)OH | 4.3 |
| Val⁸-Tyr¹⁶-Trp¹⁹-Glu²²-GLP-1(7-37)OH | 3.5 |
| Val⁸-Trp¹⁶-Glu²²-Val²⁵-Ile³³-GLP-1(7-37)OH | 5.0 |
| Val⁸-Trp¹⁶-Glu²²-Ile³³-GLP-1(7-37)OH | 4.1 |
| Val⁸-Glu²²-Val²⁵-Ile³³-GLP-1(7-37)OH | 4.9, 5.8, 6.7 |
| Val⁸-Trp¹⁶-Glu²²-Val²⁵-GLP-1(7-37)OH | 4.4 |

1. A method of treating gastroparesis in a patient suffering therefrom which comprises administering to said patient an effective amount of a GLP-1 compound.

2. The method of claim 1 wherein the patient also suffers from diabetes.

3. The method of claim 2 wherein the patient suffers from type 2-diabetes.

4. The method of any one of claims 1 to 3 wherein the GLP-1 compound is selected from the group consisting of GLP-1(7-36)amide, GLP-1(7-37), GLP-1 analogs, and GLP-1 derivatives.

5. The method of any one of claims 1 to 3 wherein the GLP-1 compound is selected from the group consisting of: Exendin-4, Exendin-4 analogs, and Exendin-4 derivatives.

6. The method of any one of claims 1 to 3 wherein the GLP-1 compound is an agonist of the GLP-1 receptor.

7. The method of any one of claims 1 to 3 wherein the GLP-1 compound is a DPP-IV resistant analog.

8. The method of claim 7 wherein the GLP-1 compound is a GLP-1 analog or derivative having glutamic acid at position 22.

9. The method of claim 7 wherein the GLP-1 compound is a GLP-1 analog having valine or glycine at position 8.

10. The method of any one of claims 1 to 3 wherein the GLP-1 compound is a GLP-1 derivative.

11. The method of claim 10 wherein the GLP-1 derivative is an acylated GLP-1 analog.

12. The method of claim 11 wherein the GLP-1 derivative is Arg³⁴Lys²⁶-(N-ε-(γ-Glu(N-α-hexadecanoyl)))-GLP-1(7-37).

13. The method of any one of claims 1 to 12 wherein the GLP-1 compound is administered by subcutaneous injection.

14. The method of any one of claims 1 through 12 wherein the GLP-1 compound is administered orally.

15. The method of any one of claims 1 through 12 wherein the GLP-1 compound is administered by buccal administration.

16. (cancelled)

17. (cancelled)
